(19) United States
(12) Patent Application Publication
(10) Pub. No.: US 2026/0241003 A1
(43) Pub. Date: Aug. 20, 2026

(54) ALL-IN-ONE MULTIMODAL NANOTHERANOSTIC PLATFORM FOR IMAGE-GUIDED THERAPY

(71) Applicant: The Johns Hopkins University, Baltimore, MD (US)

(72) Inventors: Jeff W.M. BULTE, Baltimore, MD (US); Ali SHAKERI-ZADEH, Baltimore, MD (US); Chao WANG, Baltimore, MD (US)

(21) Appl. No.: 18/876,853

(22) PCT Filed: Jul. 7, 2023
(86) PCT No.: PCT/US2023/027106

§ 371 Date: Dec. 19, 2024

Related U.S. Application Data
(60) Provisional application No. 63/359,687, filed on Jul. 8, 2022.
(60) Provisional application No. 63/397,682, filed on Aug. 12, 2022.

(51) Int. Cl. A61K 41/00; A61K 49/00; A61K 49/04; A61K 49/18; A61K 49/22

(52) U.S. Cl. CPC A61K 41/0052; A61K 41/0038; A61K 49/0002; A61K 49/0428; A61K 49/183; A61K 49/1833; A61K 49/1869; A61K 49/222

(57) ABSTRACT

A theranostic nanocomplex comprising albumin, bismuth sulfide (Bi₂S₃), and superparamagnetic iron oxide (SPIO) within a single nanoplatform and its use for tracking a cell, imaging a cell, and/or treating a disease, condition, or disorder is disclosed.

### CROSS-REFERENCE TO RELATED APPLICATIONS

This application is a national stage application under 35 U.S.C. § 371 of PCT/US2023/027106, filed Jul. 7, 2023, which claims priority to U.S. Provisional Application No. 63/359,687, filed on Jul. 8, 2022, and U.S. provisional Application No. 63/397,682, filed Aug. 12, 2022, the contents of which are herein incorporated by reference.

### FEDERALLY SPONSORED RESEARCH OR DEVELOPMENT

This invention was made with government support under EB028904 awarded by the National Institutes of Health. The government has certain rights in the invention.

### BACKGROUND

A large variety of nanoparticles have been developed for magnetic resonance imaging (MRI), magnetic particle imaging (MPI), magneto-motive ultrasound imaging (MMUS), (magneto) photoacoustic imaging ((m) (PAI)), and computed tomography (CT). At present, each imaging modality needs its own optimized magnetic or radiopaque (X-ray) contrast agent. Thus, there is a need for multimodal theranostic agents.

### SUMMARY

In some aspects, the presently disclosed subject matter provides a nanocomplex comprising albumin, bismuth sulfide (Bi₂S₃), and a superparamagnetic iron oxide (SPIO) particle coated with a natural or semisynthetic polymer, or other polymer, including a dendrimer and/or a polypeptide.

In certain aspects, the albumin comprises a serum albumin. In particular aspects, the serum albumin is selected from bovine serum albumin (BSA), human serum albumin (HSA), and recombinant HSA.

In certain aspects, the natural or semisynthetic polymer is selected from dextran, carboxydextran, a polyglucose sorbitol carboxymethyl ether, and a dendrimer, also referred to as a “dendron.”

In certain aspects, the SPIO is selected from a ferumoxide, ferucarbotran, and ferumoxytol.

In certain aspects, the nanoplex further comprises a targeting agent. In particular aspects, the targeting agent is selected from folic acid (FA), an antibody and fragments thereof, a growth factor, a vitamin, a lipid, a carbohydrate, a cancer targeting ligand, a protein, a nucleic acid aptamer, a peptide, a glycoprotein, and a glycolipid.

In particular aspects, the cancer targeting ligand comprises a sugar. In more particular aspects, the cancer targeting ligand comprises glucose. In particular aspects, the protein comprises transferrin.

In certain aspects, the nanoplex further comprises a cell. In more certain aspects, the cell is a stem cell, progenitor cell, precursor cell, or an immune cell. In particular aspects, the stem cell comprises a human mesenchymal stem cell (hMSC).

In certain aspects, the nanocomplex has a particle size having a range from about 50 to about 250. In particular aspects, the particle size is about 90 nm.

In certain aspects, the nanoplex has a ratio of albumin and Bi₂S₃ to SPIO of between about 10:1 to about 1:1. In particular aspects, the nanoplex has a ratio of albumin and Bi₂S₃ to SPIO of 10:1, 5:1, or 1:1. In yet more particular aspects, the nanoplex has a ratio of albumin and Bi₂S₃ to SPIO of about 5:1.

In other aspects, the presently disclosed subject matter provides a method for tracking a cell, the method comprising administering a presently disclosed nanocomplex to a subject or a cell and monitoring a location of the cell. In certain aspects, the cell is a stem cell, progenitor cell, precursor cell, or an immune cell. In certain aspects, the location comprises a tumor, a brain disease, and a myocardial infarction. In certain aspects, the tracking is in vivo. In certain aspects, the tracking is in vitro.

In particular aspects, the method comprises monitoring the location of the cell by MPI, CT, MPI/CT, MRI, MPI/MRI, MMUS, (m)PAI, and combinations thereof.

In further aspects, the method further comprises measuring an efficiency of cell delivery, an amount of migration of the cell, or a biodistribution of the cell in a tissue.

In other aspects, the presently disclosed subject matter provides a method for diagnosing a disease, condition, or disorder, the method comprising administering a presently disclosed nanoplex comprising a targeting agent to a subject having or suspected of having the disease, condition, or disorder and obtaining an image. In certain aspects, the image comprises an MPI, MRI, MMUMS, (m)PAI or a CT image. In certain aspects, the method allows an accurate co-registration of an MPI signal with anatomical CT imaging. In certain aspects, the method allows an allows an accurate cell quantification with both MPI and CT.

In other aspects, the presently disclosed subject matter provides a method for treating a disease, condition, or disorder, the method comprising administering a presently disclosed nanoplex comprising a targeting agent to a subject in need of treatment thereof.

In certain aspects, the method further comprises irradiating the nanoplex. In certain aspects, the method further comprises irradiating the nanoplex with an optical laser to induce photothermal heating of the tissue, subjecting the nanoplex to an alternating magnetic field to induce magnetic fluid hyperthermia (MFH) of the tissue, or treating the nanoplex with high-intensity focused ultrasound (HIFU) to induce sonothermal heating of the tissue. In particular aspects, the method further comprises irradiating the nanoplex with a gamma irradiator. In yet more particular aspects, the nanoplex acts as a radiosensitizer. In even yet more particular aspects, the method comprises targeted image-guided cancer therapy.

In some aspects, the method further comprises simultaneous or sequentially tracking a cell, imaging a cell, and/or treating a subject with the currently disclosed nanoplex.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 demonstrates adjusting the hydrodynamic size of AB comprising BSA and Bi₂S₃ by controlling the incubation time;

FIG. 2 demonstrates adjusting the size of AB by controlling the acid:base ratio;

FIG. 3A shows AF4-MALS/DLS analysis of AB nanocomplex showing the increase in shape factor and size/size distribution (MALS and DLS) after human plasma incubation indicating protein binding to the surface;

FIG. 3B shows AF4-MALS/DLS analysis of ABS nanocomplex showing the increase in shape factor and size/size distribution (MALS and DLS) after human plasma incubation indicating protein binding (forming of a corona) to the surface.

FIG. 4 shows the UV-Vis spectra (left panel) and CT effect of 1.5 mg/mL (right panel) of different AB formulations;

FIG. 5 shows the FTIR spectra of AB (left panel) and TEM images of AB (right panel);

FIG. 6 shows the characterization of ABS. Particle size of AB particles (left panel) and ABS particles (right panel);

FIG. 7 shows the HR-TEM and elemental mapping of ABS;

FIG. 8 shows the UV-Vis spectra of FA, AB, ABS, ABS-FA at 10 μg/mL; and SS at 2 μg/mL. At 808 nm: Abs_{(ABSFA)}>Abs_{(ABS)}>Abs_{(AB)};

FIG. 9 shows representative FTIR spectra of SPIO, BSA-Bi₂S₃ (AB), BSA-Bi₂S₃-SPIO (ABS), FA, and BSA-Bi₂S₃-SPIO-FA (ABS-FA);

FIG. 10 is CT of AB in solution demonstrating the effect of AB concentration on the HU value;

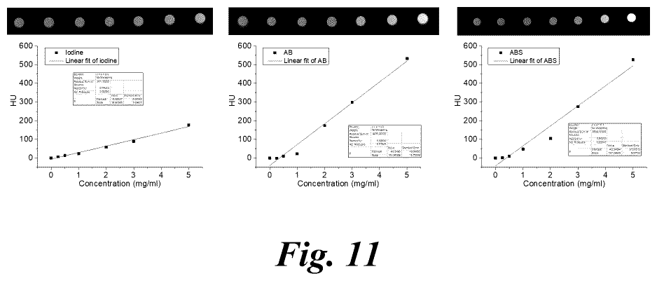

FIG. 11 is CT of AB/ABS in solution demonstrating the CT effect of Iopamidol, AB, and ABS;

FIG. 12 shows CT of AB/ABS-labeled hMSCs;

FIG. 13 shows MPI of ABS in solution;

FIG. 14 shows PB staining of AB-labeled hMSCs;

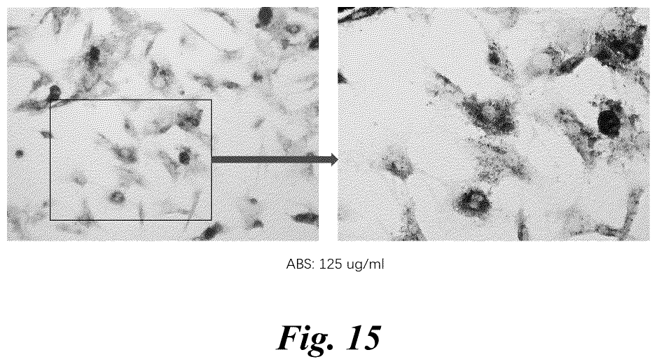

FIG. 15 shows PB staining of ABS-labeled hMSCs;

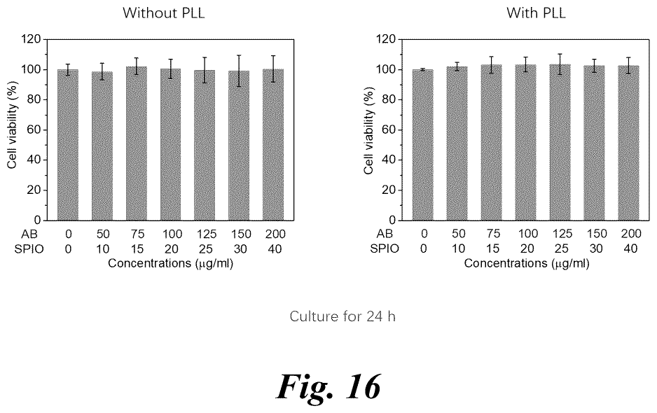

FIG. 16 shows cell viability of AB-labeled hMSCs. LDH test of hMSC incubated with AB without PPL (left panel) and with PPL (right panel) (culture for 24 h);

FIG. 17 shows cell viability of ABS. LDH test of hMSCs incubated with ABS incubated for 24 h (left panel) and 48 h (right panel);

FIG. 18 shows MPI of FA-ABS-labeled PCa cells (left panel) and CT of FA-ABS-labeled PCa cells (right panel);

FIG. 19 shows the in vitro photothermal effect of ABS. Concentration of ABS: 0.2 ml/mL; laser wavelength: 808 nm; laser power density: 1.5 W/cm²; irradiation time: 5 min. The temperature increase from 21.2° C. to 63.4° C. in 5 min; ΔT is 42.2° C.;

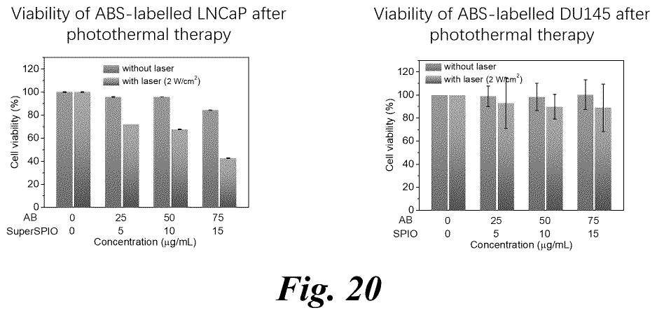

FIG. 20 shows the in vitro photothermal effect of ABS;

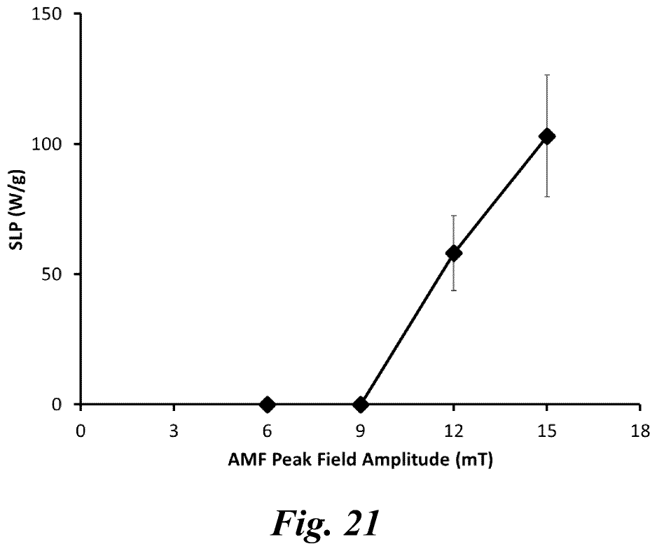

FIG. 21 demonstrates that an external alternative magnetic field (AMF)>9 mT can effectively heat the ABS complexes;

FIG. 22A, FIG. 22B, FIG. 22C, and FIG. 22D show in vivo MPI/CT of DU145 tumor bearing mice: i.t. injection. MPI/CT images of a mouse receiving naked ABS nanocomplexes (FIG. 22A) 30 minutes and (FIG. 22B) 48 hours after i.t. injection; FIG. 22C and FIG. 22D show MPI/CT images of a mouse receiving ABS-labeled hMSCs, 30 minutes and 48 hours after i.t. injection, respectively;

FIG. 22E shows in vivo MPI/CT of DU145 tumor bearing mice with 1E6 ABS-labeled hMSCs via i.v. injection;

FIG. 22F shows ex vivo MPI/CT of DU145 tumors. Ex vivo MPI/CT data along with a photograph of all three tumors are shown (top: 1E5 ABS-hMSC injected i.t.; middle: naked ABS injection i.t.; bottom: 1E6 ABS-hMSC injected i.v.);

FIG. 23A and FIG. 23B demonstrate that combined dual-contrast MPI/CT imaging of intra-cerebrally injected ABS-labeled hMSCs allows accurate anatomical co-registration of MPI signal and cell quantification using both imaging modalities;

FIG. 24 shows in vivo magnetic particle imaging (MPI) and dynamic signal analysis of ABS-labeled MSCs that were injected into the brain striatum of Rag2−/− mice;

FIG. 25 shows serial in vivo 3D MPI and MRI imaging of mice receiving ABS-MSCs intracerebrally;

FIG. 26 shows quantitative in vivo 3D CT imaging 30 days post-injection; and

FIG. 27 shows imaging and histology data of a mouse receiving 100K ABS-MSCs intracerebrally.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### ABBREVIATIONS

AB: Albumin-Bi₂S₃
ABS: Albumin-Bi₂S₃-SPIO
BSA: Bovine serum albumin
CT: Computed tomography
DLS: Dynamic light scattering
EDC: (1-ethyl-3-(3-dimethylaminopropyl) carbodiimide
EDTA: Ethylenediaminetetraacetic acid
FA: Folic acid
FTIR: Fourier transform infrared
HIFU: High-intensity focused ultrasound
HSA: Human serum albumin
hMSC: Human mesenchymal stem cell
HR-TEM: High resolution transmission electron microscopy
HU: Hounsfield unit
LDH: Lactic dehydrogenase
mPAI: magneto-photoacoustic imaging
MFH: Magnetic fluid hyperthermia
MSOT: Multispectral optoacoustic tomography
MPI: Magnetic particle imaging
MMUS: Magneto-motive ultrasound imaging
NP: Nanoparticle
PAI: Photoacoustic imaging
PCa: Prostate cancer
PLL: Poly-L-lysine
PSMA: Prostate-specific membrane antigen
SC: Subcutaneous
SPIO: Superparamagnetic iron oxide

The presently disclosed subject matter provides a new hybrid nanoprobe comprising albumin, Bi₂S₃, and SPIO particles (the combination referred to herein as “ABS”), as a new all-in-one MRI, MPI, MMUMS, (m)PAI and CT multimodal contrast or therapeutic agent.

The SPIO component allows visualization with MRI, MPI MMUS, and mPAI. The bismuth sulfide component not only provides CT and PAI image contrast, but also acts as a sensitizer for radiotherapy, photothermal therapy, HIFU and MFH, making it a unique combined trimodal diagnostic/therapeutic (i.e., theranostic) agent.

The presently disclosed ABS can be targeted to specific tumors or used for labeling of cells that home to tumors. Since the particles are visible on the imaging scans, a laser (for photothermal therapy), an alternating magnetic field for MFH or gamma ray (for radiotherapy) beam can be precisely aimed at the nanoparticles only in the tumor, minimizing off-target damage to the surrounding normal tissue.

In some embodiments, the presently disclosed subject matter provides a nanocomplex comprising albumin, Bi₂S₃, and a SPIO particle coated with a natural or semisynthetic polymer, or other polymer, including a dendrimer and/or a polypeptide.

In certain embodiments, the albumin comprises a serum albumin. In particular embodiments, the serum albumin is selected from BSA, HSA, and rHSA.

In certain embodiments, the natural or semisynthetic polymer is selected from dextran, carboxydextran, a polyglucose sorbitol carboxymethyl ether, and a dendrimer, also referred to as a “dendron.”

In certain embodiments, the SPIO is selected from a ferumoxide, ferucarbotran, and ferumoxytol.

In certain embodiments, the nanoplex further comprises a targeting agent. In particular embodiments, the targeting agent is selected from folic acid (FA), an antibody and fragments thereof, a growth factor, a vitamin, a lipid, a carbohydrate, a cancer targeting ligand, a protein, a nucleic acid aptamer, a peptide, a glycoprotein, and a glycolipid. In particular embodiments, the cancer targeting ligand comprises sugar. In more particular embodiments, the cancer targeting ligand comprises glucose. In particular embodiments, the protein comprises transferrin.

In certain embodiments, the nanoplex further comprises a cell. In more certain embodiments, the cell is a stem cell, progenitor cell, precursor cell, or an immune cell. In particular embodiments, the stem cell comprises a hMSC.

In certain embodiments, the nanocomplex has a particle size having a range from about 50 to about 250. In particular embodiments, the particle size is about 90 nm.

In certain embodiments, the nanoplex has a ratio of albumin and Bi₂S₃ to SPIO of between about 10:1 to about 1:1. In particular embodiments, the nanoplex has a ratio of albumin and Bi₂S₃ to SPIO of 10:1, 5:1, or 1:1. In yet more particular embodiments, the nanoplex has a ratio of albumin and Bi₂S₃ to SPIO of about 5:1.

In other embodiments, the presently disclosed subject matter provides a method for tracking a cell, the method comprising administering a presently disclosed nanocomplex to a subject or a cell and monitoring a location of the cell. In certain embodiments, the cell is a stem cell, progenitor cell, precursor cell, or an immune cell. In certain embodiments, the location comprises a tumor, a brain disease, and a myocardial infarction. In certain embodiments, the tracking is in vivo. In certain embodiments, the tracking is in vitro.

In particular embodiments, the method comprises monitoring the location of the cell by MPI, CT, MPI/CT, MRI, MPI/MRI, MMUS, (m)PAI, and combinations thereof.

In further embodiments, the method further comprises measuring an efficiency of cell delivery, an amount of migration of the cell, or a biodistribution of the cell in a tissue.

In certain embodiments, the method allows an accurate co-registration of an MPI signal with anatomical CT imaging. In certain embodiments, the method allows an allows an accurate cell quantification with both MPI and CT.

In other embodiments, the presently disclosed subject matter provides a method for diagnosing a disease, condition, or disorder, the method comprising administering a presently disclosed nanoplex comprising a targeting agent to a subject having or suspected of having the disease, condition, or disorder and obtaining an image. In certain embodiments, the image comprises an MPI, MRI, MMUS, (m)PAI or a CT image.

In further embodiments, the method further comprises measuring an efficiency of cell delivery, an amount of migration of the cell, or a biodistribution of the cell in a tissue.

In other embodiments, the presently disclosed subject matter provides a method for diagnosing a disease, condition, or disorder, the method comprising administering a presently disclosed nanoplex comprising a targeting agent to a subject having or suspected of having the disease, condition, or disorder and obtaining an image. In certain embodiments, the image comprises an MPI, MRI, MMUS, (m)PAI or a CT image.

In other embodiments, the presently disclosed subject matter provides a method for treating a disease, condition, or disorder, the method comprising administering a presently disclosed nanoplex comprising a targeting agent to a subject in need of treatment thereof.

In certain embodiments, the method further comprises irradiating the nanoplex. In certain embodiments, the method further comprises irradiating the nanoplex with an optical laser to induce photothermal heating of the tissue, subjecting the nanocomplex to an alternating magnetic field for MFH, or subjecting the nanocomplex to HIFU for sonothermal heating of the tissue. In particular embodiments, the method further comprises irradiating the nanoplex with a gamma irradiator. In yet more particular embodiments, the nanoplex acts as a radiosensitizer. In even yet more particular embodiments, the method comprises targeted image-guided cancer therapy.

In some embodiments, the method further comprises simultaneous or sequentially tracking a cell, imaging a cell, and/or treating a subject with the currently disclosed nanoplex.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

The term “combination” is used in its broadest sense and means that a subject is administered only one agent, more particularly an ABS nanoparticle in combination with an applied therapeutic modality, e.g., photothermal therapy, MFH, or HIFU. More particularly, the term “in combination” refers to the concomitant administration of an agent and an applied therapeutic modality for the treatment of a single disease state. As used herein, the active agent or applied therapeutic modality may be combined and administered at the same time, or may be administered alternately or sequentially on the same or separate days.

Further, the presently disclosed ABS nanoparticle in combination with an additional therapeutic modality can be further administered with adjuvants that enhance stability of the agents, alone or in combination with the agent, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed ABS nanoparticle in combination with the additional applied therapeutic modality can be varied so long as the beneficial effects of the combination of the agent and additional therapeutic modality are achieved. Accordingly, the phrase “in combination with” refers to the administration of an ABS nanoparticle described herein and an additional therapeutic modality either simultaneously, sequentially, or a combination thereof. Therefore, a subject administered a combination of a presently disclosed ABS nanoparticle and an additional therapeutic modality can receive an ABS nanoparticle and additional therapeutic modality at the same time (i.e., simultaneously) or at different times (i.e., sequentially, in either order, on the same day or on different days), so long as the effect of the combination of the agent and additional therapeutic modality is achieved in the subject.

When administered sequentially, the agent and additional therapeutic modality can be administered within 1, 5, 10, 30, 60, 120, 180, 240 minutes or longer of one another. In other embodiments, the agent and additional therapeutic modality can be administered sequentially, can be administered within 1, 5, 10, 15, 20 or more days of one another.

When administered in combination, the effective dosage of the agent to elicit a particular biological response may be less than the effective dosage of the agent when administered alone, thereby allowing a reduction in the dose of the agent relative to the dose that would be needed if the agent was administered as a single agent. The effects of the agent along with the additional therapeutic modality, but need not be, additive or synergistic. The agent and/or the additional therapeutic modality may be administered multiple times.

In some embodiments, when administered in combination, the agent and the additional therapeutic modality can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

BSA-Bi₂S₃-SPIO (ABS) Nanoparticles
1.1. Synthesis and Characterization of BSA-Bi₂S₃-SPIO (ABS)
1.1.1. Synthesis of BSA-Bi₂S₃ (AB)

Bovine serum albumin (BSA)-Bi₂S₃ (AB) nanoparticles (NP) were synthesized by a biomineralization method using BSA, Bi(NO₃)₃, HNO₃, and NaOH.

More particularly, BSA was added to double-distilled (dd) H₂O to form a first solution. Bi(NO₃)₃. H₂O was added to HNO₃, followed by the addition of ddH₂O to form a second solution. NaOH was dissolved in ddH₂O to form a third solution.

The second solution comprising the Bi salt was added to the first solution comprising BSA with ultrasound. The third solution comprising NaOH is then added with stirring followed with centrifuging to provide AB nanoparticles. The AB nanoparticles can be washed with ddH₂O, e.g., repeat washing 3×.

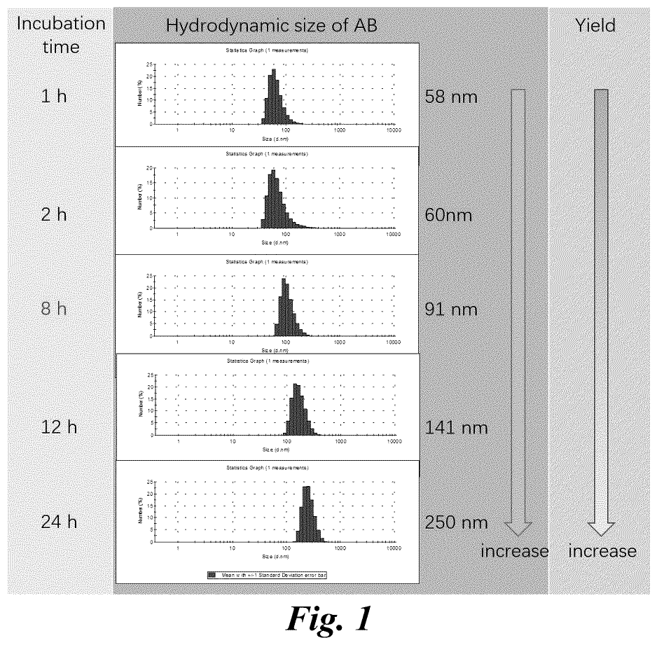

First, the incubation time to make the AB NPs was optimized. Longer incubation times provided larger particle sizes and higher yields (see FIG. 1). 8 hours was selected as the incubation time for an optimized size and yield, leading to a AB NP formulation having a hydrodynamic size of approximately 90 nm.

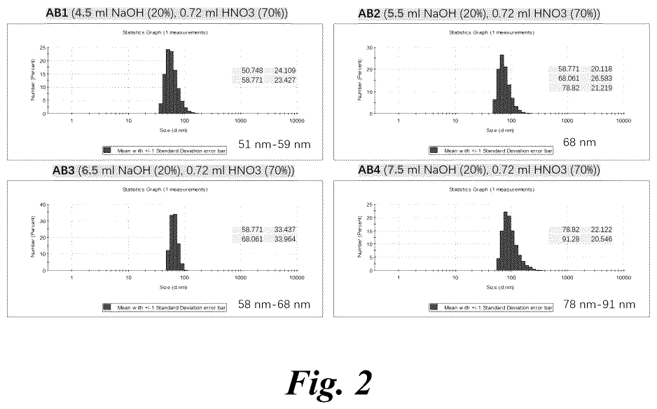

Second, the AB formulation was optimized by adjusting the ratio of NaOH and HNO₃. Larger particle sizes and a better CT effect (expressed in HU values) were observed as more NaOH was added (see FIG. 2). A volume of 7.5 ml NaOH was selected to make optimum AB NPs based on the HU values obtained in CT imaging studies.

Further physicochemical characterization of nanocomplexes was performed by the NIH Nanotechnology Characterization Laboratory (NCL). Zeta potential, hydrodynamic size, and particle concentration for the AB nanocomplex were determined to be −20.9 mV, 87 nm, and 6.4E12 particles per mL, respectively. For the ABS nanocomplex the respective values were −24.2 mV, 120 nm, and 2.9E12 particles per mL (1 mg Fe/mL or 17.9 μmol). Hence, each ABS nanocomplex contains 3,695,000 iron atoms.

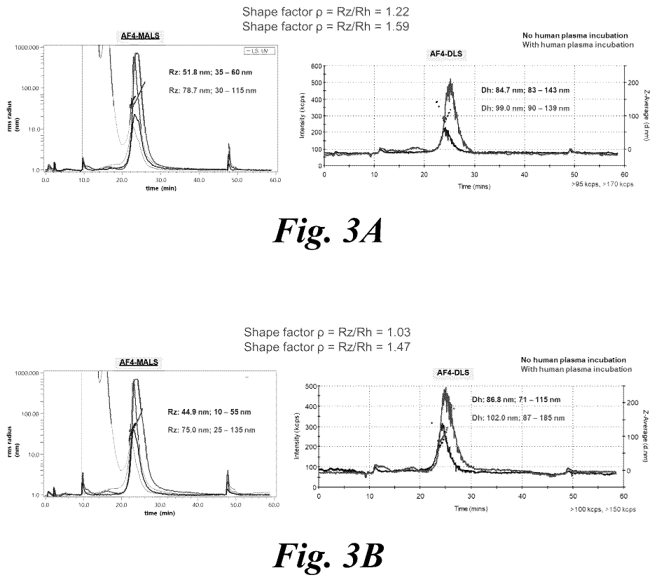

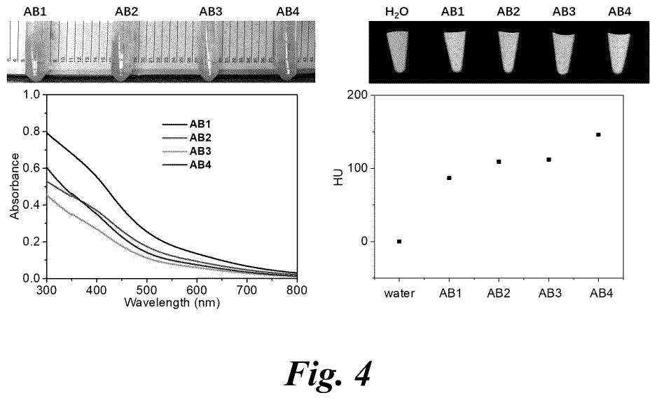

Referring now to FIG. 3A and FIG. 3B, asymmetric Flow Field-Flow Fractionation (AF4)-Multi-Angle Light Scattering (MALS)/Dynamic Light Scattering (DLS) analysis was performed to study the protein corona formation on the surface of both AB (FIG. 3A) and ABS (FIG. 3B) after incubation with human plasma.

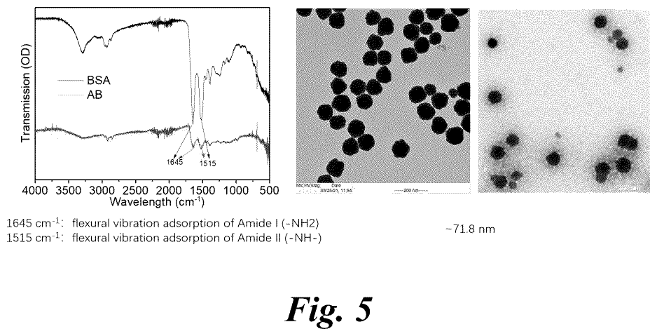

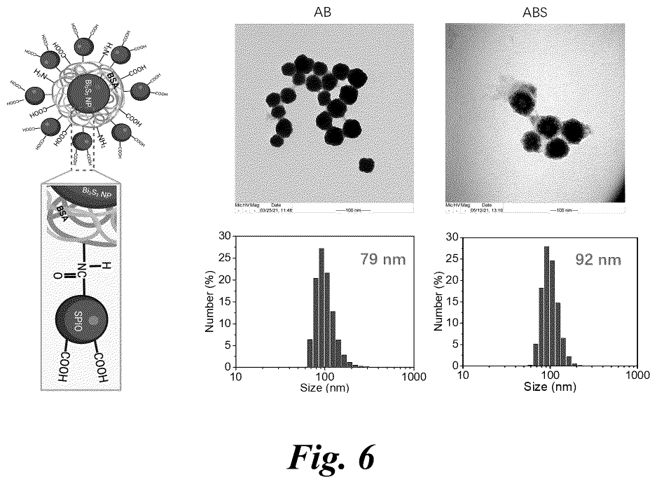

FIG. 4 shows the UV-Vis spectra (left panel) and CT effect of ABa at 1.5 m/mL (right panel) of AB. FIG. 5 shows the FTIR spectra of AB (left panel) and TEM images of AB (right panel).

### 1.1.2. Superparamagnetic Iron Oxide (SPIO) Particles

Different SPIO formulations were tested, including commercially purchased RESOVIST® (ferucarbotran (iron oxide particles coated with carboxydextran), available from Bayer Healthcare), nanoflowers, and SuperSPIO20 provided under an MTA with the Université de Franche-comté and SuperBranche, respectively. Since BSA has both NH₂ and COOH functional groups in its structure, it should be possible to use any SPIO formulation (either with negative or positive surface charge) to make the presently disclosed ABS NPs. Other SPIO nanoparticles include, but are not limited to, Ferumoxtran-10 (COMBIDEX®, AMAG Pharma; SINEREM®, Guerbet), NC100150 (CLARISCAN®, Nycomed), (VSOP C184, Ferropharm), Magtrace™ (Endomag), Sentimag® (Endomag), Synomag® (MicroMod), Perimag® (Micromod), Nanomag® (Micromod), Ferucarbotran (Resovist®, Meito-Sangyo), FeraTrack® (Miltenyi Biotec), and Ferumoxytol (FERAHEME®).

1.1.3. Synthesis of BSA-Bi₂S₃-SPIO (ABS)

BSA-Bi₂S₃-SPIO (ABS) NPs were synthesized by mixing AB and SPIO in the presence of ethylenediaminetetraacetic acid (EDTA) or 1-ethyl-3-(3-dimethylaminopropyl) carbodiimide (EDC), washing with ddH₂O and centrifugation to obtain the final product. It is possible to use any mass ratio of AB:SPIO, but the ultimate mass ratio selected depends on the eventual application. Formulations were made with different ratios of AB: SPIO and the NPs were tested with MPI. A higher MPI signal intensity was observed with a higher amount of SPIO. A ratio of 5:1 was selected based on data for cellular uptake and cytotoxicity of ABS NPs.

### 1.1.4. Synthesis of FA-BSA-BizS3-SPIO (FA-ABS)

30-mg ABS was mixed with 0.6-mg folic acid (FA) and stirred overnight at room temperature to fabricate FA-ABS NPs.

### 1.1.5. Characterization of ABS

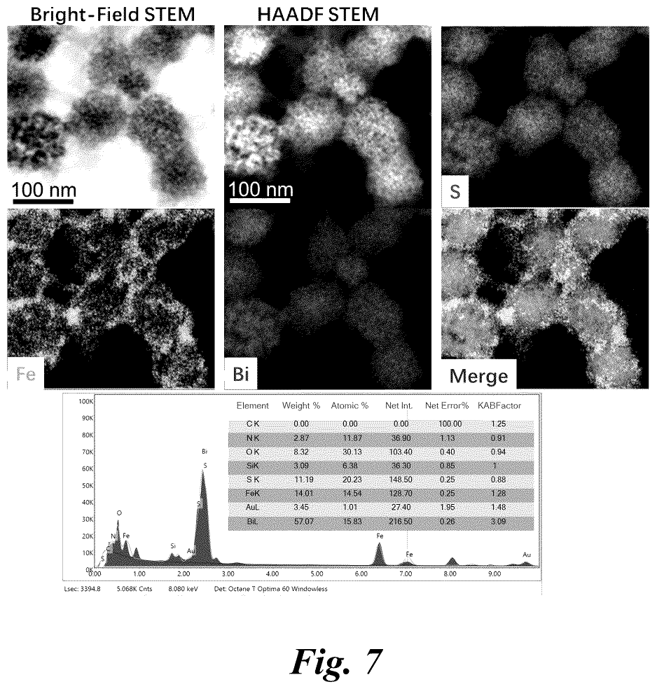

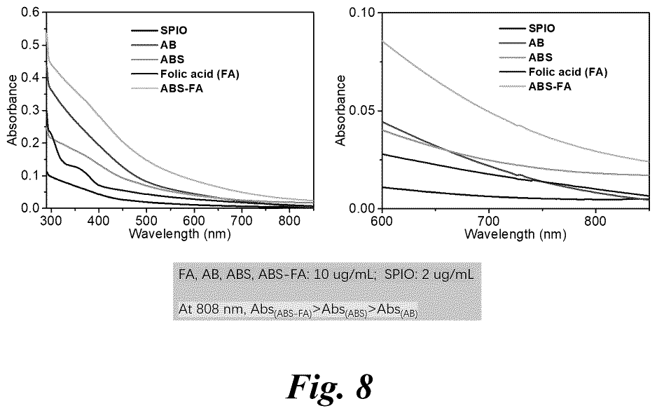

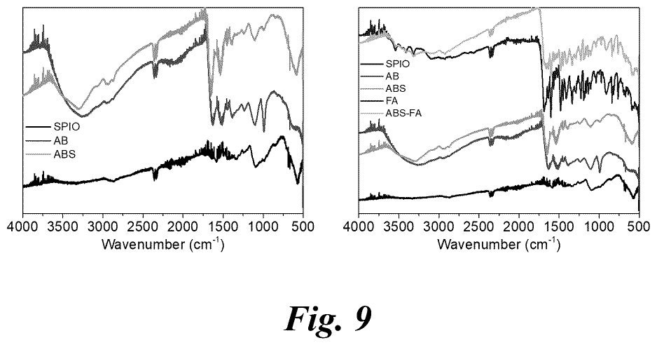

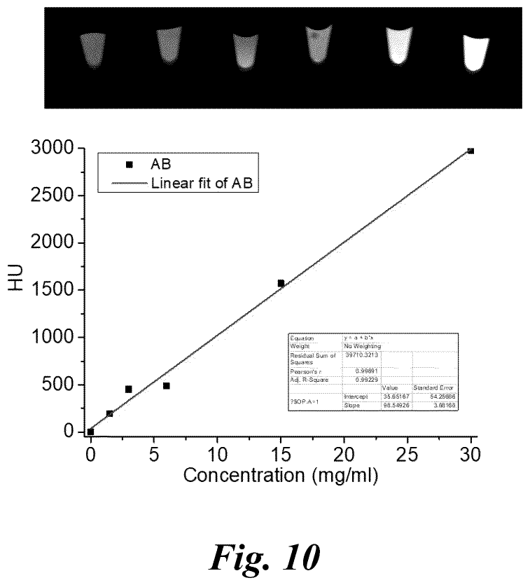

DLS (FIG. 6), HR-TEM with elemental mapping (FIG. 7), UV-Vis spectrophotometry (FIG. 8), and FTIR spectrometry (FIG. 9) were used to characterize the synthesized AB, ABS, and FA-ABS NPs.

### 1.2. In Vitro Tests of ABS

### 1.2.1. Cytotoxicity of AB and ABS NPs

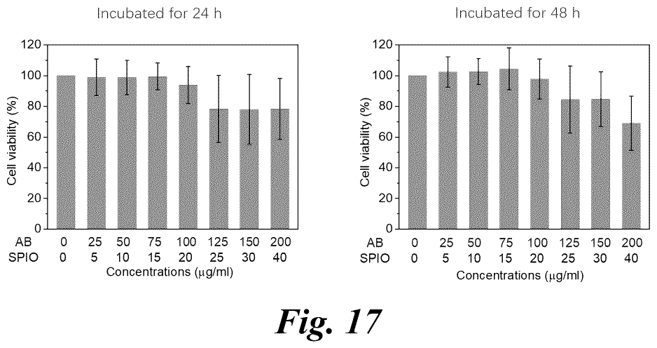

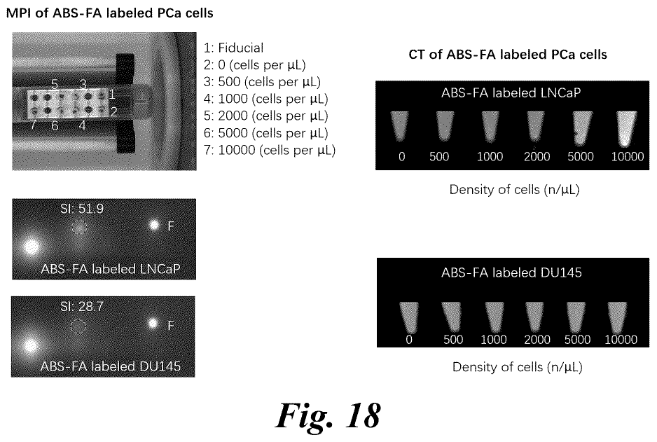

hMSCs were used to test the cytotoxicity of AB and ABS NPs using an LDH kit. hMSCs were labeled with and without PLL as a transfection agent. AB NPs had no significant cytotoxicity even when cells were incubated with 200 μg/mL for 24 h (see FIG. 16). For ABS, the hMSCs were incubated with NPs for 24 h or 48 h. Cell toxicity was negligible when the concentration of ABS was below 125 μg Bi/mL (or the equivalent of 25 μg Fe/mL) (see FIG. 17).

### 1.2.2. Cellular Uptake of AB and ABS NPs

The cellular uptake of AB and ABS NPs was evaluated by incubating them with hMSCs for 24 hours. Different concentrations of AB NPs were tested; the higher concentration, the more uptake. NPs were internalized and found within the cytoplasm with a peri-nuclear distribution, which is typical for endocytosis of nanoparticles. Based on the cell uptake and cytotoxicity data, 125 μg Bi or 25 μg Fe per mL was selected as an optimum concentration for further studies.

### 1.3. In Vitro Imaging

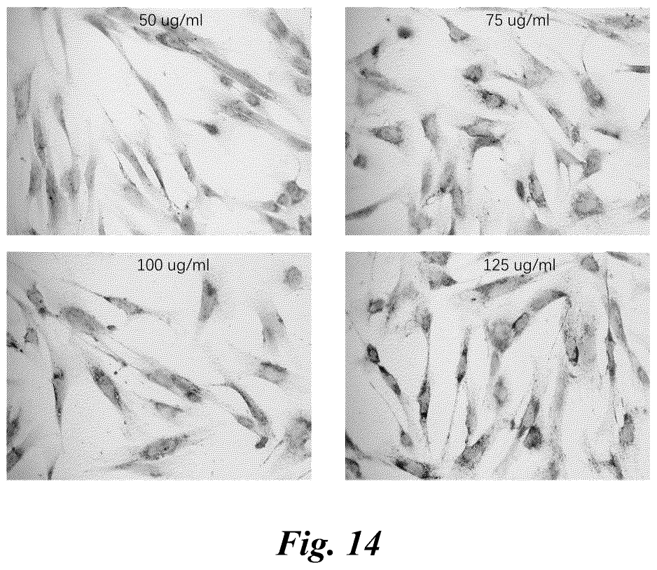

ABS with different ratios of AB: SPIO (10:1, 5:1, 1:1) were tested for their MPI imaging properties. The more SPIO, the stronger the MPI signal intensity (FIG. 13).

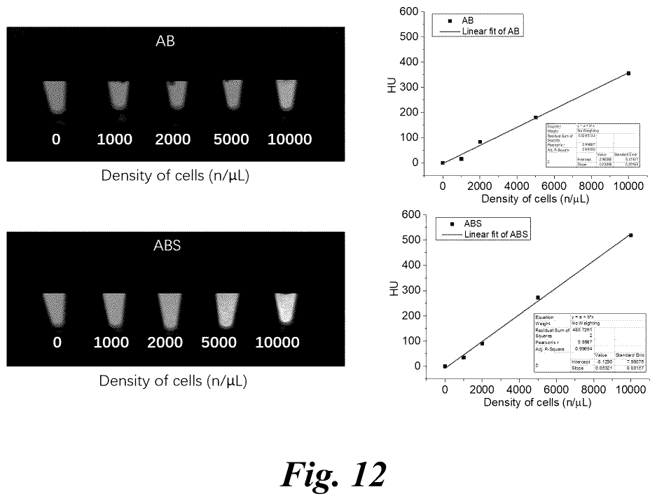

The CT effect of Iopamidol (clinical formulation, used as reference gold standard), AB, and ABS was evaluated at different concentrations. AB and ABS had similar CT contrast and both gave HU values higher than Iopamidol at the same concentrations (see FIG. 11).

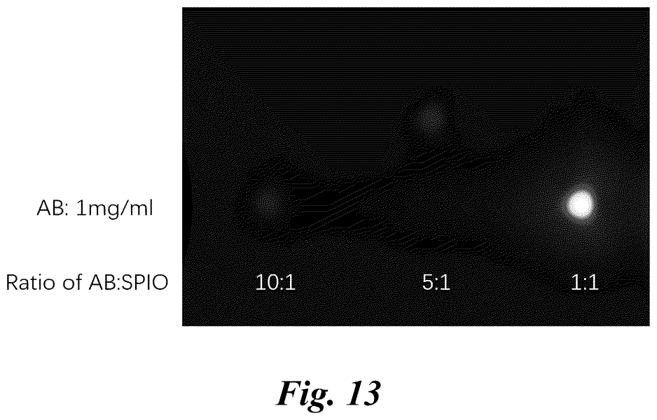

AB/ABS-labeled hMSCs were collected and dispersed in Eppendorf tubes. A HU value of approximately 520 was obtained for 10,000 ABS-labeled hMSCs per μL (see FIG. 12).

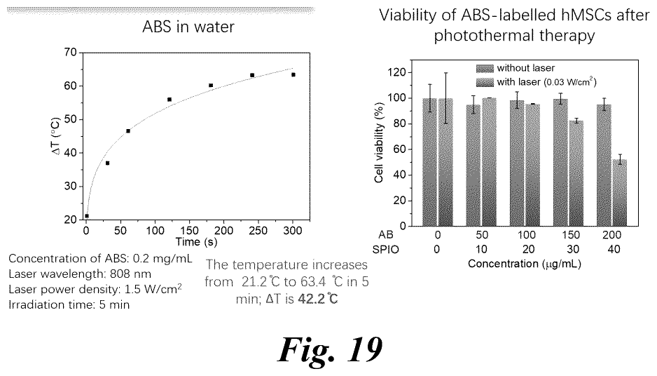

FA-ABS NPs also were incubated with two prostate cancer cells (LNCaP and DU145) with different levels of PSMA expression. PSMA has been recognized as a receptor FA, which can lead to shuttling of FA-conjugated NPs into the cells. Labeled PCa cells were evaluated with CT/MPI. A major difference was detected between the CT/MPI signals obtained for LNCaP (high expression of PSMA) and DU145 (low expression of PSMA), showing that tumor targeting is possible using an appropriate receptor/target. See FIG. 18.

1.4. In Vivo Cell Tracking of ABS-Labeled Cells with MPI/CT

### 1.4.1. In Tumor

Naked ABS or ABS-labeled hMSCs were injected directly into SC DU145 tumors and MPI/CT was performed in vivo 30 min and 48 h after injection. ABS-labeled hMSCs also were injected intravenously and then MPI/CT was performed in vivo 2 h and 24 h after injection. Tumors were taken out and ex vivo MPI/CT of all tumors was performed for further investigations.

### 1.4.2. In Brain

hMSCs were labeled with ABS and injected directly into the brain. The labeled cells could be detected in the brain using CT, MRI, and MPI.

### 1.5. Photothermal Effect of ABS (for Use of ABS as Theranostic Nanoparticles)

### 1.5.1. In Solution

ABS (0.2 mg/mL) was irradiated with an 808-nm laser (1.5 W/cm²) for 5 min. The temperature of sample increased from 21.2° C. to 63.4° C. (a temperature change of 42.2° C.).

### 1.5.2. Cells In Vitro

hMSCs were labeled with ABS and PCa cells (DU145 and LNCaP) with FA-ABS. Labeled cells were irradiated with the laser and cell viability was measured using an LDH kit. Significant in vitro PTT effects were observed for ABS.

### 1.5.3. Cells in Vivo

FA-ABS were injected directly into a SC DU145 tumor and the tumor was irradiated with the laser 30 min after injection (exposure time: 10 min; 2 W/cm²). In another SC DU145 tumor bearing mouse, FA-ABS was injected intra-venously and the tumor was irradiated with the same laser parameters 24 h after injection. A control mouse with the same tumor model also was irradiated with laser (no FA-ABS injection). In vivo thermometry was performed using an IR camera during laser irradiation to all tumors. Significant in vivo PTT effects were observed for FA-ABS.

### 1.6 Applications

The following applications are envisioned:
(a) the presently disclosed ABS can act as a good MPI/MRI/MMUS/(m)PAI/CT agent for in vivo cell tracking of hMSCs. Hence, the presently disclosed formulation can be used for monitoring stem cell therapy or immune cell therapy in living individuals, in terms of cell delivery, migration, and tissue biodistribution;
(b) PSMA-overexpressing PCa cancer cells selectively bind FA-ABS. Hence, the presently disclosed formulation can be used for cancer diagnosis. Virtually any other tumor-specific ligand can be conjugated to ABS instead of FA;
(c) the presently disclosed ABS can be used as a photothermal sensitizer if irradiated with a laser;
d) the presently disclosed ABS can be used as an MFH agent if subjected to an alternating magnetic field;
(e) the presently disclosed ABS can be used as a radiosensitizer when using a gamma irradiator for radiation therapy, and;
(f) the presently disclosed ABS can be used as a sonothermal sensitizer when using HIFU.

Accordingly, the presently disclosed ABS are both a diagnostic agent and a therapeutic agent, i.e., a nanotheranostic agent detectable by MPI, MRI, MMUS, (m)PAI and CT with potential for targeted image-guided cancer therapy.

### 1.7 Summary

In summary, a new theranostic nanocomplex made of albumin, e.g., BSA, as a matrix to incorporate both Bi₂S₃ and SPIO within a single nanoplatform have been synthesized. Different methods to optimize the ABS formulation in terms of different applications were tested, including MPI/CT and PTT.

### Example 2

### Magnetic/Radiopaque Nanoparticles for In Vivo MPI/MRI/CT Imaging and Tracking of Mesenchymal Stem Cells as a Delivery Vehicle for Cancer Treatment

### 2.1 Background and Scope

Passive and active targeting of therapeutic nanoparticles (NPs) toward cancer cells have not been entirely successful and efforts are being made to establish an effective targeting strategy that would ensure uniform distribution of the NPs throughout the tumor. The use of stem cells having inherent trophic properties for homing to tumors has been proposed as a new approach for NP delivery to cancer cells. (Su et al., 2021; Cheng et al., 2019; Wang et al., 2019). Monitoring the homing and intratumoral distribution of transplanted cells, as well as off-target site biodistribution in the rest of the body, is highly desirable. In vivo hybrid imaging has potential to meet this demand (Srivastava et al., 2014; Bulte, 2019). To this end, the presently disclosed subject matter provides a bimodal cell tracking method using a novel superparamagnetic radiopaque nanocomplex that can be detected with MPI, MRI, and CT.

### 2.2 Methods and Materials

Through a step-by-step solvothermal decomposition method, a ABS nanocomplex composed of bovine serum albumin (BSA), radiopaque Bi₂S₃ nanoparticles and superparamagnetic iron oxide (SPIO) was fabricated. ABS nanocomplexes were characterized with different techniques. hMSCs were labeled with poly-L-lysine as a secondary transfection agent and the ABS nanocomplexes for 24 hours. Naked ABS or ABS-labeled hMSCs were injected intratumorally (i.t.) or intravenously (i.v.) in DU145 (human prostate cancer)-bearing mice. Thirty minutes and 48 hours after injection, mice were imaged with MPI and CT. Two days after i.t. injection or four days after i.v. injection, mice were sacrificed and tumors were excised for ex vivo imaging.

### 2.3 Results

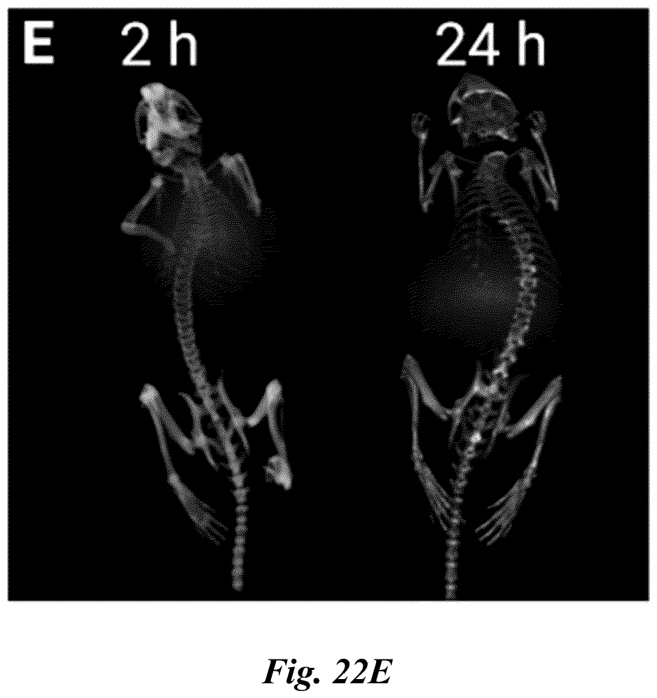

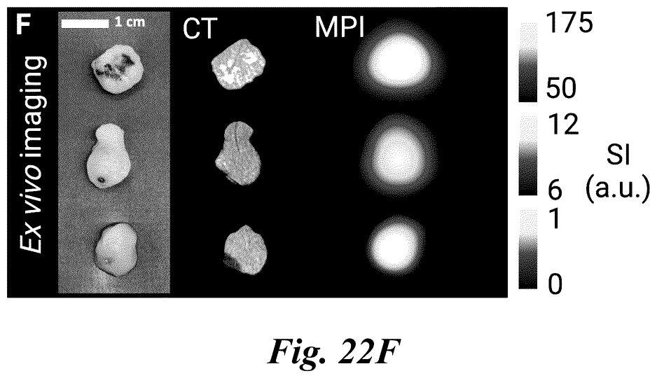

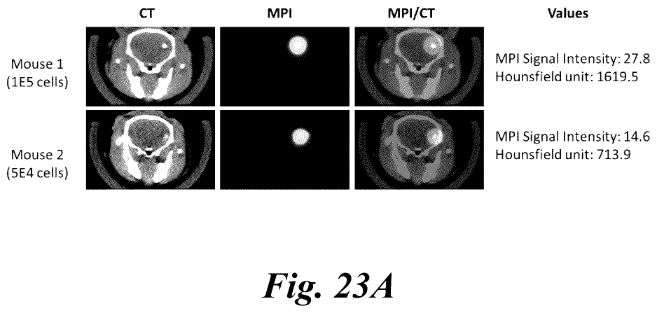

Referring now to FIG. 7, the ABS nanocomplexes prepared immediately hereinabove exhibited a spherical morphology with an even distribution of bismuth, iron, and sulfur across the spheres (average size: 90 nm). In vivo MPI/CT images of mice receiving naked ABS nanocomplexes or ABS-hMSCs after i.t. injection are shown in FIG. 22A-22D. The i.t. injection of ABS-hMSCs demonstrated that labeled cells moved throughout the entire tumor and maintained a strong signal intensity over 48 hours, while naked ABS nanocomplexes remained at a focal point near the injection site and exhibited a decreasing signal intensity.

Referring now to FIG. 22E for i.v. injection, homing of cells to the lung was observed 2 hours after injection and in the liver 24 hours later. No signal could be observed in the tumor for i.v. injection. Ex vivo imaging showed the highest amount of MPI signal intensity was obtained for i.t. injection of ABS-hMSCs (FIG. 22F).

### 2.4 Conclusion

The presently disclosed subject matter demonstrates the feasibility of in vivo bimodal imaging of naked ABS and ABS-labeled hMSCs using CT and MPI and the disparity in imaging between the naked ABS and ABS-labeled hMSCs. Protocols for MPI/MRI/CT-guided hyperthermal therapy using stem cell delivery of ABS nanocomplexes are currently being developed.

### Example 3

### All-In-One Superparamagnetic Radiopaque Nanocomplex for In Vivo MRI, MPI, and CT Stem Cell Tracking

### 3.1 Background

Clinical trials using stem cells as a regenerative treatment are on the rise, but in many cases the therapeutic efficacy has been disappointing. The use of in vivo imaging techniques to track stem cell trafficking inside the body has considerable potential to enhance therapeutic outcome (Srivastava et al., 2014). One of the current limitations with in vivo cell tracking techniques, however, is that a single imaging modality cannot be responsive to all queries about the fate of transplanted cells, including cell viability, quantity, and overall biodistribution. The presently disclosed subject matter provides the development of a multi-modal cell tracking method using a novel superparamagnetic radiopaque nanocomplex for in vivo MRI, MPI, and CT tracking of MSCs, one of the most widely used therapeutic cells in humans.

### 3.2 Methods

### 3.2.1 Nanocomplex Synthesis and Characterization

Through a solvothermal decomposition method, a ABS nanocomplex was synthesized composed of BSA, radiopaque Bi₂S₃ nanoparticles and SPIO. ABS nanocomplexes were characterized with dynamic light scattering (DLS), Fourier transform infrared (FTIR) and UV-VIS spectrophotometry, and high-resolution transmission electron microscopy (HR-TEM). Elemental analysis was performed to determine the percentage of iron and bismuth in the ABS nanocomplex.

### 3.2.2 Cells

Human bone marrow-derived MSCs (P2) were obtained from Rooster Bio, USA. MSCs were incubated with ABS at a concentration of 25 μg Fe (approximately 125 μg Bi) per ml. Cell labeling was performed with and without poly-L-lysine (1125 ng/mL) as transfection agent in T-75 tissue culture flasks for 24 hours. Labeled cells were collected and prepared for injection in normal male Rag2 mice. Cell viability after incubation with ABS nanocomplex was determined using LDH assay. Prussian blue staining and a Ferrozine-based spectrophotometric assay were used to assess intracellular iron uptake.

### 3.2.3 Cell Transplantation

ABS-labeled MSCs were transplanted in the striatum of Rag2 mice under 1.5% isoflurane anesthesia. Mice were positioned in a stereotaxic device and 100,000 labeled cells in 2 μL of phosphate-buffered saline were injected using a Hamilton syringe (31G, AP=0 mm, ML=2 mm, DV=3 mm, 0.5 μL/min). One hour after transplantation, mice were euthanized and heads were removed and fixed in 4% paraformaldehyde.

### 3.2.4 Imaging

A customized holder was 3D-printed for use with all MRI, MPI, and CT machines. One day post fixation, the heads were imaged ex vivo with MRI using a 17.6 T vertical bore Bruker Biospec scanner and then with MPI using a Magnetic Insight Momentum scanner. Ex vivo CT was also performed. MR images were acquired using a FLASH sequence with TR=8.4 ms, TE=2.5 ms, NEX=16, FA=5 deg, resolution=0.1 mm, slice thickness=18 mm, matrix size=150×300×180, and FOV=3×2×1.8 cm. Heads were scanned with MPI using the same FOV as MRI with 55 projections, 3D high-resolution mode, and one scan per projection. Two fiducials containing 25,000 and 50,000 labeled MSCs were placed within the MRI/MPI/CT FOVs and used for cell quantification and data co-registration using 3D slicer software.

### 3.3 Results

ABS nanocomplexes showed a spherical morphology. An even distribution of bismuth, iron and sulfur was found across the ABS spheres, with an average hydrodynamic diameter of 90 nm. The Fe: Bi ratio in ABS nanocomplex was determined as 1:5. FTIR spectra of BSA, SPIO, BSA-Bi₂S₃ nanoparticles, and the overall ABS nanocomplexes confirmed the covalent bonds between BSA, Bi₂S₃ nanoparticles and SPIOs. Prussian blue staining showed peri-nuclear accumulation of nanocomplex in labeled MSCs, with an iron content of 17 μg Fe per cell. No significant cytotoxicity was found for ABS nanocomplex. Using ex vivo imaging data, the location of transplanted cells could be easily addressed by MRI and CT, while the number of cells was quantified using MPI.

### 3.4 Discussion

The presently disclosed subject matter demonstrates the possibility of multi-modal imaging of transplanted cells with trimodal imaging using a single composite nanocomplex. In addition to visualization of cells with an anatomical context provided by CT and MRI, another benefit of using ABS nanocomplexes as a cell labeling agent is the ability to quantify cell content with MPI (Bulte 2019; Bulte et al., 2015). Since ABS is a cold tracer (without radioactivity), it also may allow easy-to-interpret whole-body distribution studies when ABS-labeled MSCs are injected systematically. Further studies are being performed in our lab to assess the effects of ABS on stem cell differentiation into adipocytes, chondrocytes and osteocytes.

### 3.5 Summary

The presently disclosed subject matter provides a novel nanocomplex for labeling and tracking stem cells using multi-modal imaging.

### Example 4

CT and MPI of ABS-Labeled hMSCs

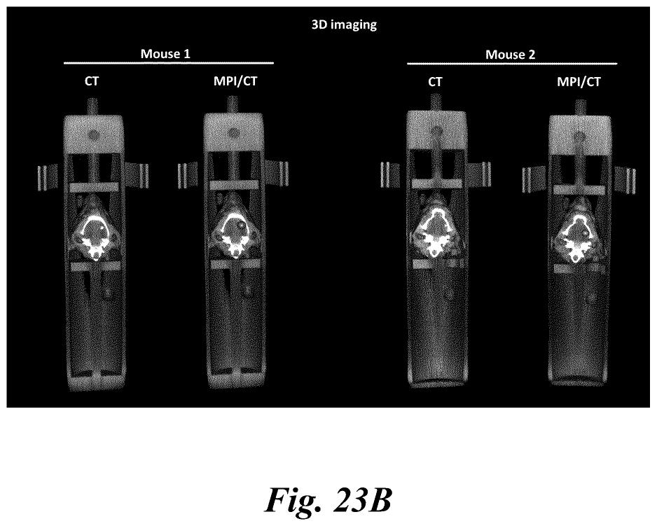

Referring to FIG. 23A and FIG. 23B are CT and MPI images of ABS-labeled hMSCs. As shown in FIG. 23A and FIG. 23B, both the CT and MPI show a perfect correlation with 2× increased signal when increasing the cell dose 2×. More particularly, FIG. 23A demonstrates that the presently disclosed methods allow accurate co-registration of “hot spot” MPI signal with anatomical CT imaging. Likewise, FIG. 23B demonstrates that the presently disclosed methods allow accurate cell quantification with both MPI and CT.

### Example 5

### Serial In Vivo Imaging of Mice Receiving ABS-MSCs Intracerebrally

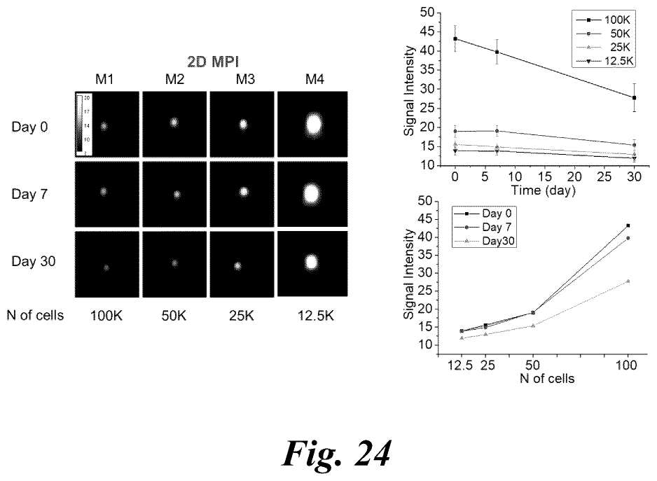

In this example, ABS-labeled MSCs (100 K cells, 50 K cells, 25 K cells, and 12.5 K cells) were injected into the brain striatum of Rag2−/− mice. The mice were imaged with micro-CT (IVIS Spectrum/CT), MRI (Bruker Biospec 9.4 T horizontal bore) and MPI (Magnetic Insight Momentum scanner) 30 min, 7 days, and 30 days after injection. Mice were then sacrificed and the fixed heads were scanned with an iThera MSOT in Vision 512-echo scanner. To validate imaging data, the brain tissues were further examined using Prussian blue and anti-HuNA staining methods. In vivo magnetic particle imaging (MPI) and dynamic signal analysis of ABS-labeled MSCx are shown in FIG. 24.

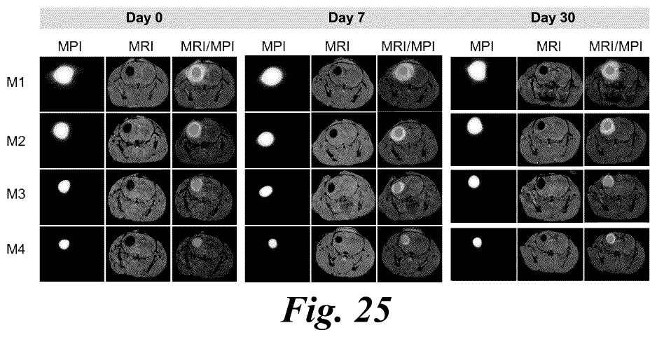

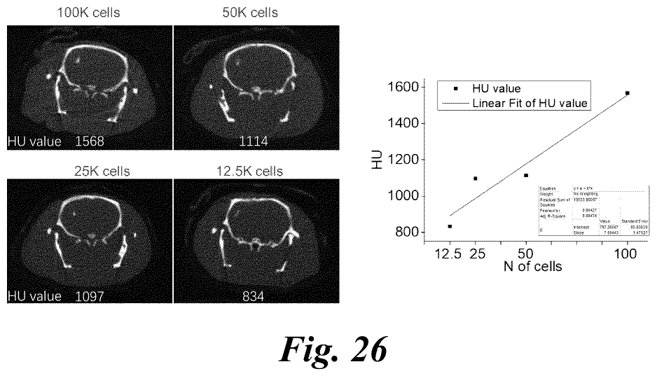

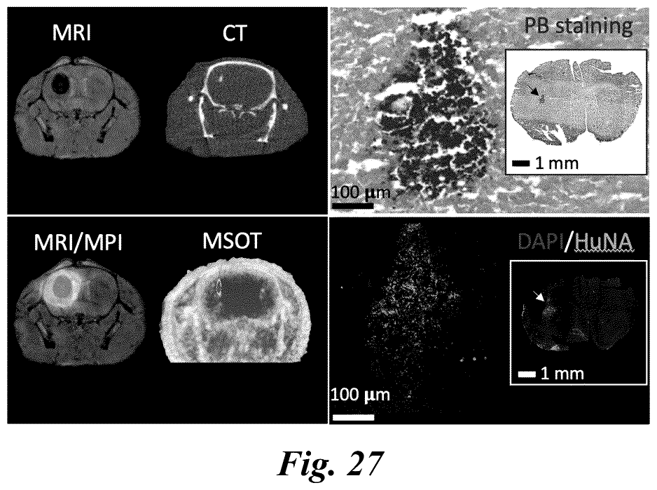

FIG. 25 shows serial in vivo 3D MPI and MRI of mice receiving ABS-MSCs intracerebrally. FIG. 26 quantitative in vivo 3D CT imaging 30 days post-injection. FIG. 27 shows imaging and histology data of a mouse receiving 100 K ABS-MSCs intracerebrally. As shown in FIG. 27, the location of transplanted cells could be easily visualized by MRI, MPI, CT, and MSOT. Prussian blue staining together with anti-HuNA staining confirmed the presence of cells containing iron in brain tissue.

### Example 6

### Magnetic Hyperthermia

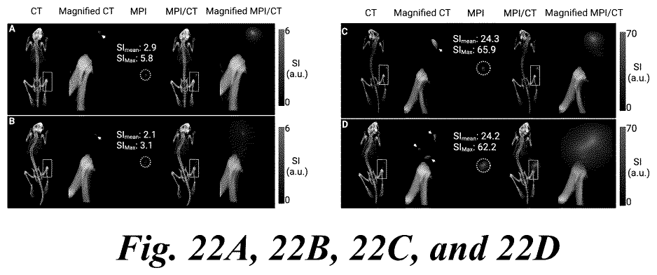

Magnetic heating properties of ABS particles within a calibrated range of 6-15 mT (4.7 kA/m-12.0 kA/m) obtained with a “HYPER” prototype instrument (Magnetic Insight, Inc.) for performing magnetic hyperthermia under MPI guidance. A “control” sample (1 mL aliquot of DI water) was placed within the HYPER at each tested field amplitude. Each sample was pulsed 20 times. A MATLAB script was used to analyze each pulse and compile an average specific loss power (SLP), as described by Carlton and Ivkov, 2023. Data provided in FIG. 21 demonstrate that an external alternative magnetic field (AMF)>9 mT can effectively heat the ABS complexes.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Bulte, J W M, et al., Quantitative “hot-spot” imaging of transplanted stem cells using superparamagnetic tracers and magnetic particle imaging. Tomography. 2015; 1 (2): 91.
Bulte J W M, Superparamagnetic iron oxides as MPI tracers: A primer and review of early applications. Adv. Drug Deliv. Rev. 2019; 138; 293.
Cheng, S, Nethi, S K, Rathi, S, Layek, B & Prabha, S. Engineered mesenchymal stem cells for targeting solid tumors: therapeutic potential beyond regenerative therapy. J. Pharmacol. Exp. Ther. 2019; 370; 231.
Srivastava A K, et al. Seeing stem cells at work in vivo. Stem Cell Rev. Rep. 2014; 10; 127.
Su Y, et al. Current advances and challenges of mesenchymal stem cells-based drug delivery system and their improvements. Int. J. Pharm. 2021; 600; 120477.
Wang, X, et al. Acta Pharm. Sin. B. 2019; 9 (1): 167.
Carlton, H and Ivkov, R, J. Appl. Physics 2023; 133 (4): 044302.

Although the foregoing subject matter has been described in some detail by way of illustration and example for purposes of clarity of understanding, it will be understood by those skilled in the art that certain changes and modifications can be practiced within the scope of the appended claims.

That which is claimed is:

1. A nanocomplex comprising albumin, bismuth sulfide (Bi₂S₃), and a superparamagnetic iron oxide (SPIO) particle coated with a natural or semisynthetic carbohydrate or other polymer, including a dendrimer and/or a polypeptide.

2. The nanocomplex of claim 1, wherein the albumin comprises a serum albumin.

3. The nanocomplex of claim 2, wherein the serum albumin is selected from bovine serum albumin (BSA), human serum albumin (HSA), and recombinant human serum albumin (rHSA).

4. The nanocomplex of claim 1, wherein the natural or semisynthetic carbohydrate is selected from dextran, carboxydextran, polyglucose sorbitol carboxymethyl ether, and a dendrimer or dendron.

5. The nanocomplex of claim 1, wherein the SPIO is selected from a ferumoxide, ferucarbotran, and ferumoxytol.

6. The nanocomplex of claim 1, further comprising a targeting agent.

7. The nanocomplex of claim 6, wherein the targeting agent is selected from folic acid (FA), an antibody and fragments thereof, a growth factor, a vitamin, a lipid, a carbohydrate, a cancer targeting ligand, a protein, a nucleic acid aptamer, a peptide, a glycoprotein, and a glycolipid.

8. The nanocomplex of claim 1, further comprising a cell.

9. The nanocomplex of claim 8, wherein the cell is a stem cell, progenitor cell, precursor cell, or an immune cell.

10. The nanocomplex of claim 9, wherein the stem cell comprises a human mesenchymal stem cell.

11. The nanocomplex of claim 1, wherein the nanocomplex has a particle size having a range from about 50 nm to about 250 nm, or a particle size of about 90 nm.

12. (canceled)

13. The nanocomplex of claim 1, wherein the nanoplex has a ratio of albumin and Bi₂S₃ to SPIO selected from between about 10:1 to about 1:1; about 10:1, 5:1, or 1:1; and about 5:1.

14-15. (canceled)

16. A method for tracking a cell, the method comprising administering a nanocomplex of claim 8 to a subject or a cell and monitoring a location of the cell.

17. The method of claim 16, wherein the cell is a stem cell, progenitor cell, precursor cell, or an immune cell.

18. The method of claim 16, wherein the location comprises a tumor, a brain disease, and a myocardial infarction.

19. The method of claim 16, wherein the tracking is in vivo or in vitro.

20. (canceled)

21. The method of claim 16, comprising monitoring the location of the cell by magnetic particle imaging (MPI), magneto-motive ultrasound imaging (MMUS), (magneto) photoacoustic imaging ((m)PAI), computed tomography (CT), MPI/CT, magnetic resonance imaging (MRI), MPI/MRI/CT and combinations thereof.

22. The method of claim 16, further comprising measuring an efficiency of cell delivery, an amount of migration of the cell, or a biodistribution of the cell in a tissue.

23. The method of claim 16, wherein the method allows an accurate co-registration of an MPI signal with anatomical CT imaging and/or an accurate cell quantification with both MPI and CT.

24. (canceled)

25. A method for diagnosing a disease, condition, or disorder, the method comprising administering a nanoplex of claim 6 comprising a targeting agent to a subject having or suspected of having the disease, condition, or disorder and obtaining an image.

26. The method of claim 25, wherein the image comprises an MPI, MRI, MMUS, (m)PAI, MSOT, or a CT image.

27. A method for treating a disease, condition, or disorder, the method comprising administering a nanoplex of claim 6 comprising a targeting agent to a subject in need of treatment thereof.

28. The method of claim 27, further comprising irradiating the nanoplex.

29. The method of claim 28, further comprising one or more of:
(a) irradiating the nanoplex with an optical laser to induce photothermal heating of the nanoplex;
(b) subjecting the nanoplex to an alternating field to induce magnetic heating of the nanocomplex;
(c) irradiating the nanoplex with a gamma irradiator; and
(d) subjecting the nanoplex to HIFU to induce sonothermal heating of the nanoplex.

30-32. (canceled)

33. The method of claim 28, wherein the nanoplex acts as a radiosensitizer.

34. The method of claim 27, comprising targeted image-guided cancer therapy.

35. A method for simultaneously or sequentially tracking a cell, imaging a cell, and/or treating a subject, the method comprising administering to the cell or subject a nanoplex of claim 1.
